(19) United States
(12) Patent Application Publication
(10) Pub. No.: US 2003/0044026 A1
(43) Pub. Date: Mar. 6, 2003

(54) Method for processing sound signal

(75) Inventor: Feng Chia Chi, Macau (CN)

Correspondence Address: ROTHWELL, FIGG, ERNST & MANBECK, P.C., 1425 K STREET, N.W., SUITE 800, WASHINGTON, DC, 20005, US

(73) Assignee: Culturecom Technology (Macau), Ltd.

(21) Appl. No.: 09/940,653

(22) Filed: Aug. 29, 2001

(51) Int. Cl. H03B 29/00; A61F 11/06

(52) U.S. Cl. 381/94.7; 381/94.1; 381/71.13

(57) ABSTRACT

A method for processing a sound signal is proposed, which is applied to a signal processing system including a signal receiving device, a signal recognizing device and a signal processing device. The signal recognizing device is used to distinguish the sound signal received from the signal receiving device into a sound source signal and background interference, so as to eliminate the background interference and transfer the sound source signal to the signal processing device. In this case, the signal processing device merely processes and amplifies the sound source signal, and thus no filtering circuit is required for the elimination of the background interference.

### FIELD OF THE INVENTION

The present invention relates to methods for processing sound signals, and more particularly, to a method for processing a sound signal, in which background interference of the sound signal is eliminated, so as to merely process a signal from a sound source for amplification.

### BACKGROUND OF INVENTION

A conventional method for elimination interference employs a filter for processing a voltage or current signal through a filtering circuit, in a manner as to allow the signal having a frequency above or below a certain frequency or within a certain frequency range to pass through the circuit, so as to distinguish the signal into a desired signal and an interfering signal. In view of operational principles, a general filter includes a low pass filter, a high pass filter, a band pass filter and an off-band pass filter. Such a filter allows a signal to be input to a circuit thereof and processes the signal to be differentiated into interference and a desired signal according to characteristics such as signal amplitude and frequency.

Accordingly, in the conventional technology, a signal can not be pre-separated into a desired component and an interfering component without being entirely processed by a filter. Therefore, it is desired to develop a method for processing a sound signal, in which, after the sound signal is distinguished into a sound source signal and background interference, the background interference is eliminated, and merely the sound source signal is processed.

### SUMMARY OF THE INVENTION

A primary objective of the present invention is to provide a method for processing a sound signal, which is applied to a signal processing system including a signal receiving device, a signal recognizing device and a signal processing device, so as to eliminate background interference and merely process a sound source signal for amplification, according to phase change and deviation of the sound signal, intensity of signal amplitude, and frequency change caused by Doppler effect.

In accordance with the foregoing and other objectives, the present invention proposes a method for processing a sound signal, which is applied to a signal processing system including a signal receiving device, a signal recognizing device and a signal processing device, and comprises the following steps.

First, the signal receiving device having a left receiving element and a right receiving element receives a signal containing a sound source signal and background interference, or selectively receives a sound signal from a particular direction by adjusting the left and right receiving elements

Then, the signal recognizing device distinguishes the received sound signal into the sound source signal and the background interference, which differ from each other in characteristics such as phase, amplitude and frequency. Therefore, the signal recognizing device makes use of phase change and deviation of the sound signal, intensity of signal amplitude, and frequency change caused by Doppler effect, so as to differentiate the sound source signal from the background interference.

After the sound source signal and the background interference are separated from each other, the signal recognizing device eliminates the background interference, and merely transfers the sound source signal to the signal processing device.

Upon receiving the sound source signal, the signal processing device processes the sound source signal for amplification, and thus no a filtering circuit is required for the elimination of the background interference.

### BRIEF DESCRIPTION OF THE DRAWINGS

The present invention can be more fully understood by reading the following detailed description of the preferred embodiments, with reference made to the accompanying drawings wherein:

FIG. 1 is a block diagram of basic system architecture for a signal processing system in the use of the method for processing a sound signal of the invention;

FIG. 2 is a schematic diagram showing the steps involved in eliminating background interference and merely amplifying a sound source signal in the signal processing system of FIG. 1 in the use of the method for processing a sound signal of the invention;

FIG. 3 is a schematic diagram showing the steps involved in determining phase change and deviation of a sound signal for eliminating background interference and merely amplifying a sound source signal in an embodiment of the signal processing system of FIG. 1 in the use of the method for processing a sound signal of the invention;

FIG. 4 is a schematic diagram showing the steps involved in determining amplitude of a sound signal for eliminating background interference and merely amplifying a sound source signal in another embodiment of the signal processing system of FIG. 1 in the use of the method for processing a sound signal of the invention; and

FIG. 5 is a schematic diagram showing the steps involved in determining frequency change caused by Doppler effect for eliminating background interference and merely amplifying a sound source signal in a farther embodiment of the signal processing system of FIG. 1 in the use of the method for processing a sound signal of the invention.

### DETAILED DESCRIPTION OF THE PREFERRED EMBODIMENT

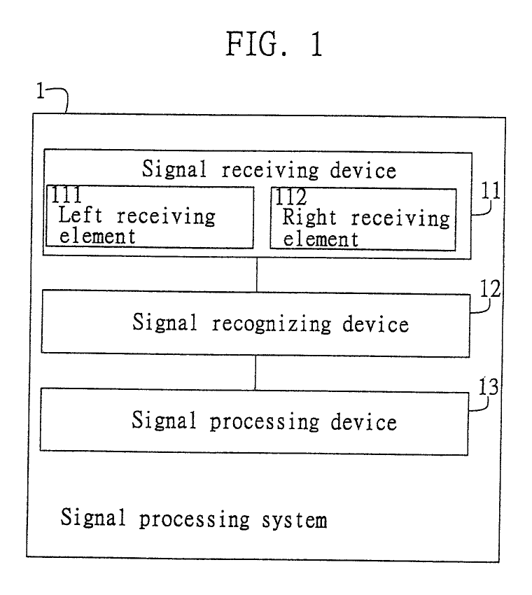

Illustrated in FIG. 1 is basic system architecture for a signal processing system in the use of the method for processing a sound signal of the invention. As shown in FIG. 1, the signal processing system 1 includes a signal receiving device 11, a signal recognizing device 12 and a signal processing device 13, wherein the signal receiving, device 11 is coupled to the signal recognizing device 12, which is further coupled to the signal processing device 13.

The signal receiving device 11 has a left receiving element 111 and a right receiving element 112, so as to receive a sound signal containing a sound source signal and background interference, or to selectively receive a sound signal from a particular direction by adjusting the left and right receiving elements 111, 112. The signal receiving device 11 transfers the received sound signal to the signal recognizing device 12, which is used to distinguish the received sound signal into the sound source signal and the background, according to phase change and deviation of the sound signal, intensity of signal amplitude, and frequency change caused by Doppler effect. The signal recognizing device 12 then eliminates the background interference, and merely transfers the sound source signal to the signal processing device 13. Subsequently, the sound source signal is processed by the signal processing device 13 for amplification, and thus it is not necessary to employ a filtering circuit for eliminating the background interference.

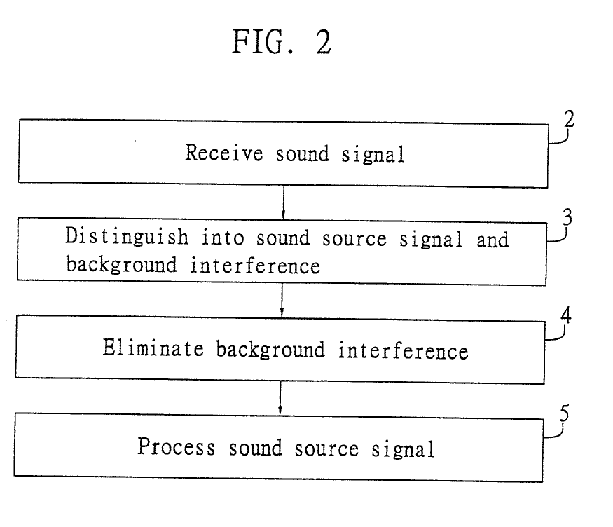

FIG. 2 illustrates the steps involved in using the method for processing a sound signal of the invention for the signal processing system of FIG. 1.

As shown in the drawing, first in step 2, the signal receiving device 11 having the left receiving element 111 and the right receiving element 112 receives a signal containing a sound source signal and background interference, or selectively receives a sound signal from a particular direction by adjusting the left and right receiving elements 111, 112. Then step 3 is followed.

In step 3, the signal recognizing device 12 distinguishes the received sound signal into the sound source signal and the background interference, which differ from each other in characteristics such as phase, amplitude and frequency. Therefore, the signal recognizing device 12 makes use of phase change and deviation of the sound signal, intensity of signal amplitude, and frequency change caused by Doppler effect, so as to differentiate the sound source signal from the background interference.

In step 4, after the sound source signal and the background interference are separated from each other, the signal recognizing device 12 eliminates the background interference, and merely transfers the sound source signal to the signal processing device 13. Then step 5 is followed.

In step 5, upon receiving the sound source signal, the signal processing device 13 processes the sound source signal for amplification, without using a filtering circuit for eliminating the background interference.

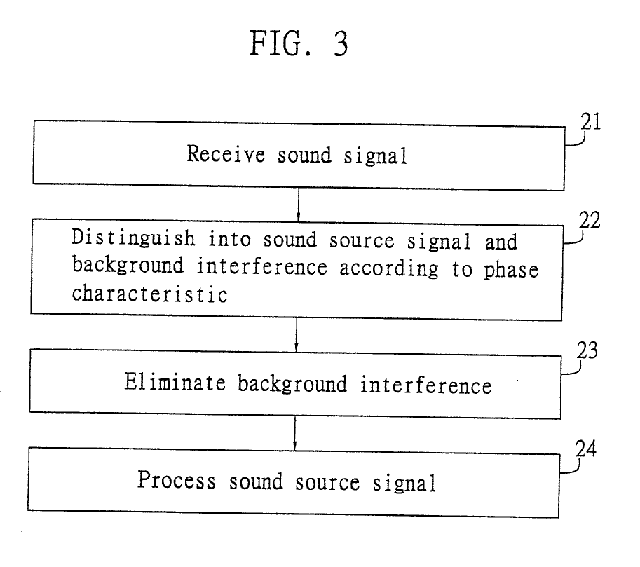

FIG. 3 illustrates the steps involved in determining phase change and deviation of a sound signal for eliminating background interference and merely amplifying a sound source signal in an embodiment of the signal processing system of FIG. 1 in the use of the method for processing a sound signal of the invention.

First in step 21, the signal receiving device 11 having the left receiving element 111 and the right receiving element 112 receives a signal containing a sound source signal and background interference, or selectively receives a sound signal from a certain (particular) direction by adjusting the left and right receiving elements 111, 112 according to phase difference for the received sound signal (e.g. phase lead or delay). Then step 22 is followed.

In step 22, the signal recognizing device 12 distinguishes the received sound signal into the sound source signal and the background interference in response to phase difference therebetween. Therefore, the signal recognizing device 12 makes use of phase change and deviation of the sound signal so as to differentiate the sound source signal from the background interference.

In step 23, after the sound source signal and the background interference are separated from each other, the signal recognizing device 12 eliminates the background interference, and merely transfers the sound source signal to the signal processing device 13. Then step 24 is followed.

In step 24, upon receiving the sound source signal, the signal processing device 13 processes the sound source signal for amplification, without using a filtering circuit for eliminating the background interference.

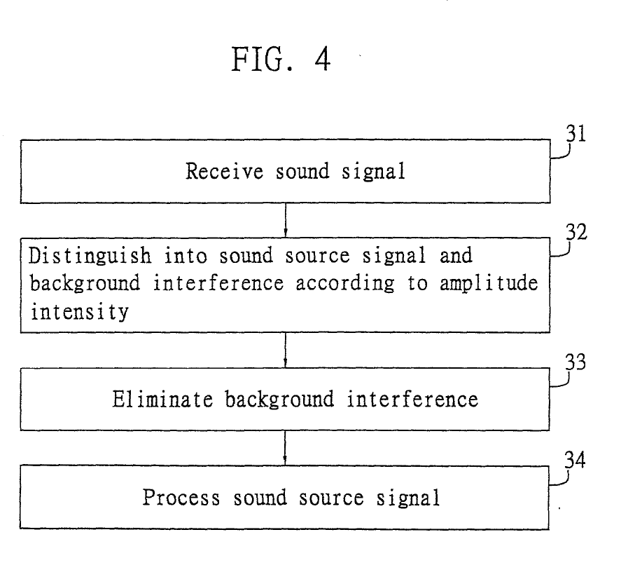

FIG. 4 illustrates the steps involved in determining amplitude of a sound signal for eliminating background interference and merely amplifying a sound source signal in another embodiment of the signal processing system of FIG. 1 in the use of the method for processing a sound signal of the invention.

First in step 31, the signal receiving device 11 having the left receiving element 111 and the right receiving element 112 receives a signal containing a sound source signal and background interference, or selectively receives a sound signal from a particular direction by adjusting the left and right receiving elements 111, 112. Then step 32 is followed.

In step 32, the signal recognizing device 12 distinguishes the received sound signal into the sound source signal and the background interference in response to amplitude difference therebetween. Therefore, the signal recognizing device 12 makes use of intensity of signal amplitude so as to differentiate the sound source signal from the background interference.

In step 33, after the sound source signal and the background interference are separated from each other, the signal recognizing device 12 eliminates the background interference, and merely transfers the sound source signal to the signal processing device 13. Then step 34 is followed.

In step 34, upon receiving the sound source signal, the signal processing device 13 processes the sound source signal for amplification, without using a filtering circuit for eliminating the background interference.

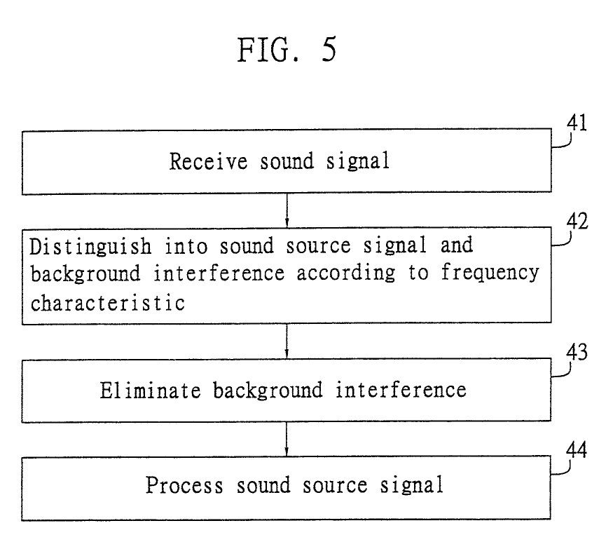

FIG. 5 illustrates the steps involved in determining frequency change caused by Doppler effect for eliminating background interference and merely amplifying a sound source signal in a further embodiment of the signal processing system of FIG. 1 in the use of the method for processing a sound signal of the invention.

First in step 41, the signal receiving device 11 having the left receiving element 111 and the right receiving element 112 receives a signal containing a sound source signal and background interference, or selectively receives a sound signal from a particular direction by adjusting the left and right receiving elements 111, 112. Then step 42 is followed.

In step 42, the signal recognizing device 12 distinguishes the received sound signal into the sound source signal and the background interference in response to velocity difference therebetween. Therefore, the signal recognizing device 12 makes use of frequency change caused by the Doppler effect so as to differentiate the sound source signal from the background interference.

In step 43, after the sound source signal and the background interference are separated from each other, the signal recognizing device 12 eliminates the background interference, and merely transfers the sound source signal to the signal processing device 13. Then step 44 is followed.

In step 44, upon receiving the sound source signal, the signal processing device 13 processes the sound source signal for amplification, without using a filtering circuit for eliminating the background interference.

In conclusion, the method for processing a sound signal of the invention is applied to the signal processing system including the signal receiving device, the signal recognizing device and the signal processing device, in which the signal recognizing device is used to distinguish the sound signal received from the signal receiving device into a sound source signal and background interference, the signal processing device. In this case, the signal processing device merely processes and amplifies the sound source signal, and thus no filtering circuit is required for the elimination of the background interference.

The invention has been described using exemplary preferred embodiments. However, it is to be understood that the scope of the invention is not limited to the disclosed embodiments. On the contrary, it is intended to cover various modifications and similar arrangements. The scope of the claims, therefore, should be accorded the broadest interpretation so as to encompass all such modifications and similar arrangements.

What is claimed is:

1. A method for processing a sound signal, applied to a signal processing system including a signal receiving device, a signal recognizing device and a signal processing device, so as to eliminate background interference and process a sound source signal for the sound signal; the method comprising the steps of:
(1) receiving the sound signal containing the sound source signal and the background interference via the signal receiving device;
(2) distinguishing the sound signal into the sound source signal and the background interference via the signal recognizing device;
(3) eliminating the background interference and merely transferring the sound source signal via the signal recognizing device to the signal processing device; and
(4) processing and amplifying the sound source signal via the signal processing device.

2. The method of claim 1, wherein in the step (2), the signal recognizing device distinguishes the sound signal into the sound source signal and the background interference according to signal phase difference.

3. The method of claim 1, wherein in the step (2), the signal recognizing device distinguishes the sound signal into the sound source signal and the background interference according to difference in signal amplitude intensity.

4. The method of claim 1, wherein in the step (2), the signal recognizing device distinguishes the sound signal into the sound source signal and the background interference according to difference in frequency change caused by Doppler effect.

5. The method of claim 1, wherein in the step (2), the signal recognizing device distinguishes the sound signal into the sound source signal and the background interference according to differences in signal phase, signal amplitude intensity, and frequency change caused by Doppler effect.

6. A method for processing a sound signal, applied to a signal processing system including a signal receiving device, a signal recognizing device and a signal processing device, so as to eliminate background interference and process a sound source signal for the sound signal; the method comprising the steps of:
(1) adjusting the signal receiving device for selectively receiving the sound signal containing the sound source signal and the background interference from a certain direction via the signal receiving device;
(2) distinguishing the sound signal into the sound source signal and the background interference via the signal recognizing device;
(3) eliminating the background interference and merely transferring the sound source signal via the signal recognizing device to the signal processing device; and
(4) processing and amplifying the sound source signal via the signal processing device.

7. The method of claim 6, wherein in the step (2), the signal recognizing device distinguishes the sound signal into the sound source signal and the background interference according to signal phase difference.

8. The method of claim 6, wherein in the step (2), the signal recognizing device distinguishes the sound signal into the sound source signal and the background interference according to difference in signal amplitude intensity.

9. The method of claim 6, wherein in the step (2), the signal recognizing device distinguishes the sound signal into the sound source signal and the background interference according to difference in frequency change caused by Doppler effect.

10. The method of claim 6, wherein in the step (2), the signal recognizing device distinguishes the sound signal into the sound source signal and the background interference according to differences in signal phase, signal amplitude intensity, and frequency change caused by Doppler effect.
